(19) United States
(12) Patent Application Publication
(10) Pub. No.: US 2026/0074065 A1
(43) Pub. Date: Mar. 12, 2026

(54) WIRELESS COMMUNICATION SYSTEM

(71) Applicants: PASCAL CLEVE, SUDBURY, MA (US); Scott Wilson, Acton, MA (US); Martin Bodley, Boston, MA (US)

(72) Inventors: PASCAL CLEVE, SUDBURY, MA (US); Scott Wilson, Acton, MA (US); Martin Bodley, Boston, MA (US)

(21) Appl. No.: 18/883,147

(22) Filed: Sep. 12, 2024

(51) Int. Cl. G16H 40/67; G10L 15/08; G10L 15/22; G10L 15/30

(52) U.S. Cl. CPC G16H 40/67; G10L 15/08; G10L 15/22; G10L 15/30; G10L 2015/088; G10L 2015/223

(57) ABSTRACT

A wireless communication system has a plurality of wireless communication devices in communication with a central hub, and the central hub is connected over a network to a caregiver and a server. Each wireless communication device has passive and active motion detection functionality, a loudspeaker, microphone, and duplex audio communication functionality. When a passive motion detector on one of the wireless communication devices detects motion, that wireless communication devices transition from an inactive to an active state, and remains in the active state while motion is detected. While in the active state, the audio communications functionality is operational and the microphone starts to capture sound.

### FIELD OF THE INVENTION

The present disclosure relates generally to patient monitoring systems, and more specifically to a patient monitoring system that operates as a proxy for a remote caregiver.

### BACKGROUND

Systems for monitoring patient status are used in a hospital environment to track a plurality of patient physiological health characteristics. These characteristics can be, for example, heart rate, blood pressure, respiratory rate, cardiovascular information, and whether or not the patient is in their bed. Typically, a variety of sensing devices are attached to the patient, or to the patient's bed, that operate to collect the physiological information that the monitoring system can then use to determine the current status of the patient's health. While these monitoring systems operate effectively in a hospital or intensive care setting to provide a high level of patient physiological information, they are generally too expensive and intrusive to be useful in a residential/home setting.

Patient health monitoring systems used in a residential setting can be of several types. One type of system, a remote monitoring system, can employ one or more networked devices to which a patient can connect to monitor detect blood pressure, heart rate, blood sugar level, etc. Subsequent to the physiological information being collected, the device can operate to transmit the information over the network (i.e., internet or phone connection) to a remote patient monitoring system for analysis. Another type of system can be comprised of a device carried by an individual that they can activate if they fall or otherwise are in trouble and need assistance. Once activated, this type to device can operate to automatically send an alert to a monitoring service that can then either notify a caregiver, a healthcare professional or local official that the patient is in trouble and needs assistance.

### BRIEF DESCRIPTION OF THE DRAWINGS

The present invention can be best understood by reading the specification with reference to the following figures, in which:

FIG. 1A illustrates functional elements comprising a residential patient monitoring system 100.

FIG. 1B illustrates a home interior with sensors placed at particular locations in different rooms.

FIG. 1C illustrates a living room 175 having two sensors positioned to detect an individual in different parts of the room.

FIG. 2 is a diagram showing functional elements comprising a multi-sensor comprising the monitoring system 100.

FIG. 3 illustrates functional elements comprising central hub 200 comprising the monitoring system 100.

FIG. 4 is a logical diagram showing the system 100 operation with one sensor in a room.

FIG. 5 is a logical diagram showing the system 100 operation with more than one sensor in a room.

### DETAILED DESCRIPTION

While it may be necessary to provide a high level of care to some hospital or residential patients using monitoring devices worn by the patients or attached to a hospital bed in which the patient is laying, not all individuals require this level of care. Further, not all individuals are comfortable, or will accept, carrying around a communication device that they can activate when they are in trouble and need assistance. In this regard, it may be sufficient to non-invasively monitor day-to-day activities, such as sleeping, eating, entertainment, or generally moving around their house. While it is possible to monitor these activities remotely by a caregiver or monitoring service using video cameras or microphones that are always on or periodically operate to send information to a monitoring service or caretaker, in the event that privacy is a concern, not all individuals agree to let others monitor their activities with video or audio monitoring devices without their knowledge or permission. In this case, other methods can be used to non-invasively collect information about the status of an individual's health and activities in their home.

Accordingly, we have designed a monitoring system that operates to non-invasively gather information about an individual's activities, while maintaining an acceptable level of privacy. Such a monitoring system can be comprised of one or more sensors, each one of which can be configured to detect multiple different types of activity. The sensors can be powered by either household alternating current (i.e., plugged into a 110 AC wall socket), or they can be battery powered, or any combination of AC and battery power. Each sensor can communicate wirelessly with a central hub, and the central hub can be connected over a wide-area network to a monitoring service and/or a caregiver. The central hub can operate to receive information from the sensors that is characteristic of one or more individual's location and activity within some defined space, and use this information to determine whether the individual is in need of assistance. If the hub determines that the individual may be in need of assistance, it can generate and send a message to either, or both of, a caregiver and a monitoring service alerting them that the individual may be in need of assistance. Further, the hub and the sensors can be configured to operate as a wireless communication device to connect the caregiver with the patient for voice calls. Still further, the hub can operate to send some or all of the sensor information to a remote server that is configured with functionality that operates intelligently to learn different types of activities (movement of sound) which are indicative of normal or abnormal behavior.

According to one embodiment, the monitoring system can operate to identify which of a plurality of sensors is in an optimal position to detect movement corresponding to one or more monitored individuals, by first employing a passive motion detection device, and then if the passive device detects movement, powering an active motion detection device.

According to another embodiment, the passive motion detection device is a passive infrared (IR) device and the active motion detection device is a millimeter wave radar device.

According to another embodiment, the powered state of each of a plurality of active sensing devices can be managed such that one device does not interfere with the operation of any of the other devices.

According to another embodiment, the monitoring system can determine a current status of one or more individuals by detecting their presence at a particular location in their home, detecting a type of motion or movement of the individual and sound detected at that location, and then using this information to determine whether or not the individual is in need of assistance.

According to another embodiment, two or more sensors can be placed in one room of a house, and each sensor can be controlled by the central hub to operate such that only a sensor in an optimal position to collect sound or movement information relating to an individual (or individuals) being monitored is active at any time. Using information collected by the sensors, the system can be trained to recognize normal patient activity, such as, but not limited to, sleeping, resting, eating, entertaining, etc., and if the information collected is indicative that the individual's activity is not normal, the system can then generate a message and send it to the caregiver alerting them that some action with respect to the individual may be necessary.

According to another embodiment, sound information can be collected by the monitoring system sensors for the purpose of classifying sound and determining whether or not an individual or individuals are behaving normally (i.e., normal activity) or not (i.e., sound is indicative that the individual may be in trouble or danger). In this regard, the system can classify sound as speech or not speech type sound. In the event the sound is classified by the system as speech, the system can recognize certain words or groupings of words, and the system can generate and send a message to the caregiver who can determine whether to contact the individual or not.

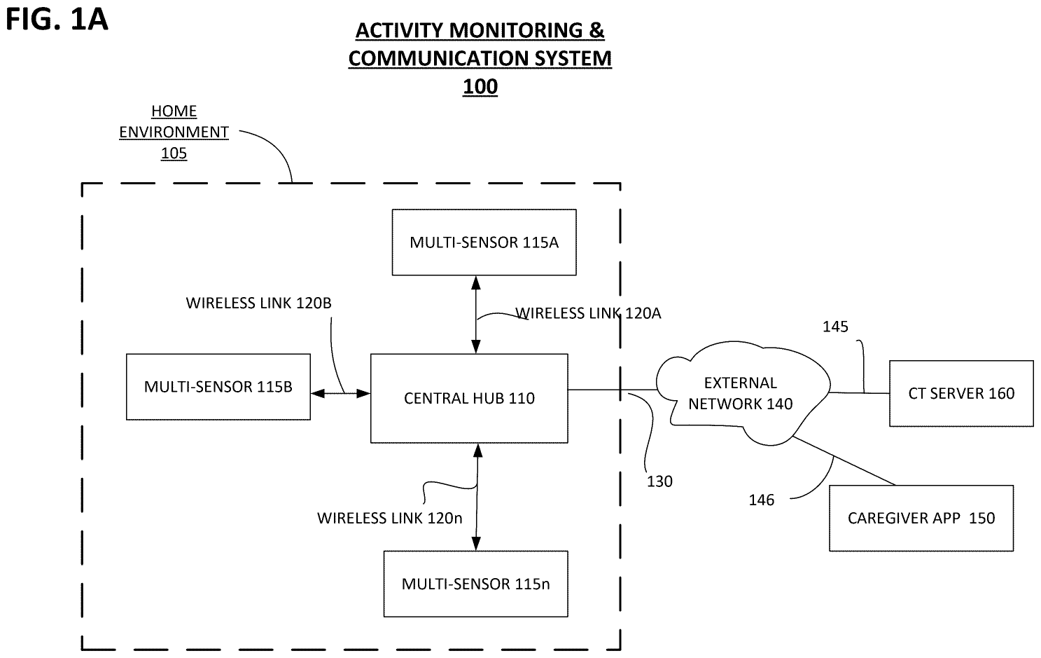

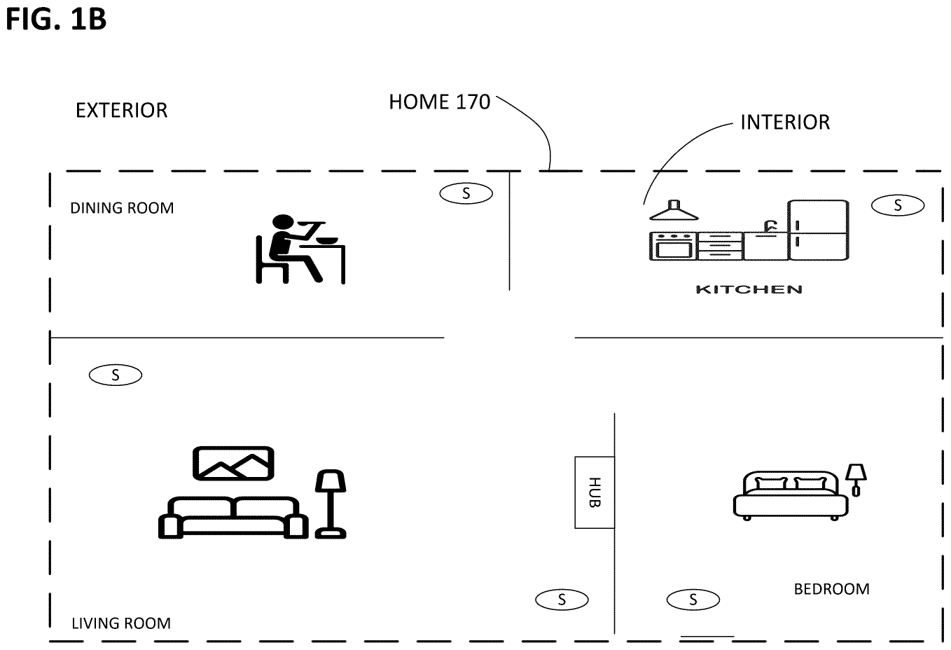

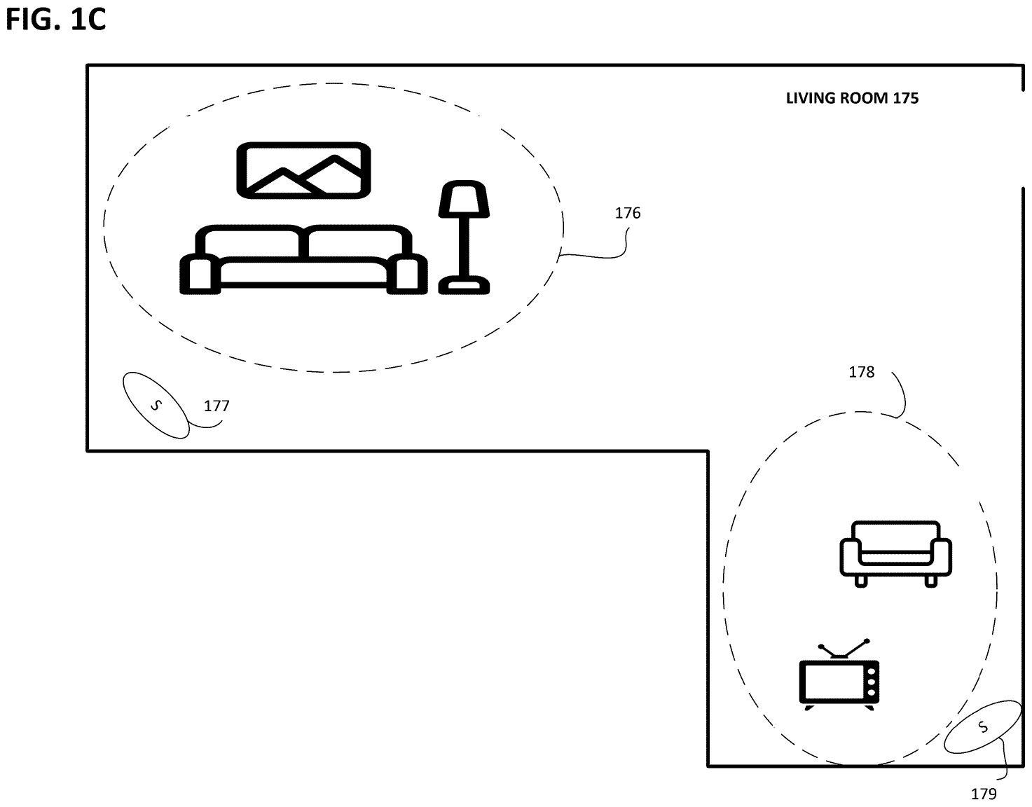

These and other embodiments are described below with reference to the drawings, in which FIG. 1A is a block diagram illustrating functional elements comprising a system 100 (monitoring system) that operates to monitor activity (i.e., movement and speech/sound activity) associated with an individual and to provide full-duplex communication between the individual and a remote caregiver. A first portion of the monitoring system 100 (local portion) can be located within a residential/home environment associate with an individual being monitored. A second portion of the system (remote portion such as caregiver application) can be located and operate at some location that is remote from the individual's residence or home. The home can include an interior space circumscribed by the dashed line 105 in FIG. 1A. It is also possible that some portion (a third portion) of the system can be located immediately outside the home, and proximate to the residence. The first portion of the system 100 located in the home's interior can comprise a central hub 110 and one or more multi-sensors (or simply sensors) 115A to 115n, with n being some integer. Each sensor is in communication with the hub 110 over a short or medium range wireless link, such as a Bluetooth or Wi-Fi connection for instance. The hub 110 is also in wireless or wired communication, over an external network 140 (i.e., Internet), with a server 160 and with a caregiver (i.e., mobile application or application running on a mobile or stationary computational device for example) both of which are typically located remotely with respect to the monitored individual's home.

Continuing to refer to FIG. 1A, the central hub 110 generally operates to receive information detected by the sensors, to process the received sensor information to determine a monitored individual's status (i.e., type of motion/movement and sound), and depending on the individual's status, whether an alert message should be generated and sent to a caregiver 150. The central hub 110, or simply hub, can be configured to control the operational state of a sensor or one or more functions comprising a sensor, and it can operate to receive messages from, and be controlled by, the caregiver 150. In this regard, the caregiver can initiate a audio session between the caregiver's communication application, the hub 110 and any one of the sensors for the purpose of listening or talking to the monitored individual to determine their status, and whether or not they need assistance.

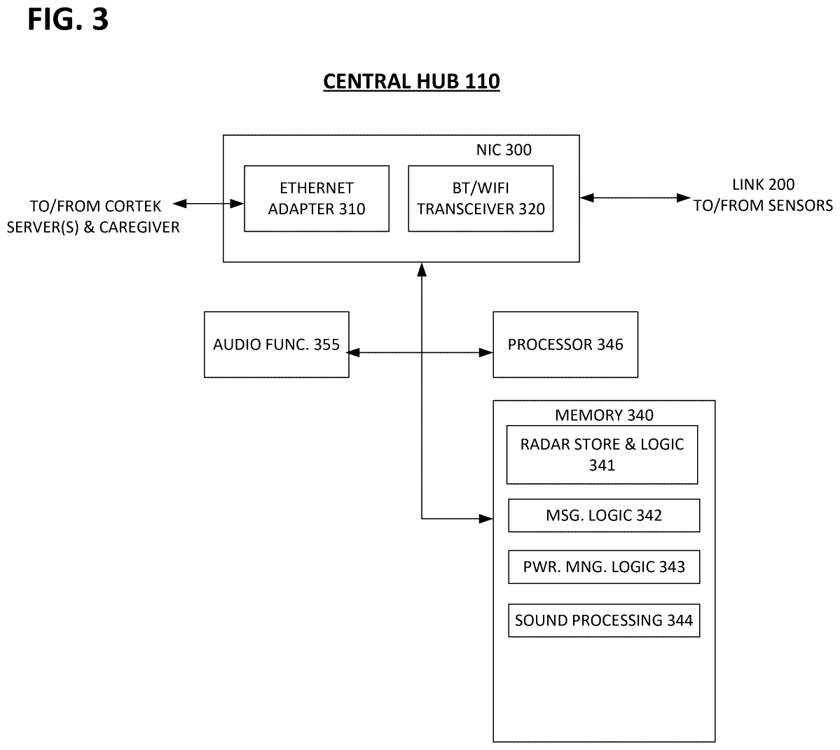

With continued reference to FIG. 1A, each of the sensors comprising the system 100 can operate to determine a current location and activity of an individual in their home. The current location of the individual can be determined based on information a sensor detects in the environment proximate to it, and at least some of this detected information can be transmitted to the hub. In this regard, each sensor has functionality that operates to passively and actively detect motion, and the actively detected motion can be sent to the hub for processing, or can be processed locally be each sensor. The motion information can be detected by one or both of a passive IR (PIR) sensor and a millimeter wave radar, the operation of which will be described later with reference to FIG. 3. Each sensor also has one or more microphones that collect sound from the home environment, and pass this sound information to the hub for processing. Based on the type of sound that the sensors receive, the system 100 can, among other things, determine whether or not to generate and send a message to a caregiver.

FIG. 1B is an illustration showing several rooms comprising a home 170 having one or more sensors in each room illustrated by a circle “S”. The sensors are placed at locations in each room to ensure that the system can detect an individual in any part of the room, and the specific placement of each sensor can be determined by the range and field-of-view (FOV) of each sensor. So, for example in FIG. 1B, each one of a dining room, kitchen, and bedroom have one sensor, while a living room (which is larger) has two sensors. Further, if the room is in in a shape, or has dimensions, that does not allow a single sensor to detect sound or movement in the room, then it may be necessary to place two or more sensors in positions such that their field of view covers the entire space. This sort of arrangement is illustrated with reference to FIG. 1C, in which a living room 175 has two separate spaces, labeled 176 and 177, that are located in different corners or areas of the room. The area labeled 176 is proximate to, and within the FOV of a sensor labeled 177, and the area labeled 178 is proximate to a sensor labeled 179. More or fewer sensors may be necessary to detect an individual in a room depending upon the size and shape of the room and the capabilities of each sensor.

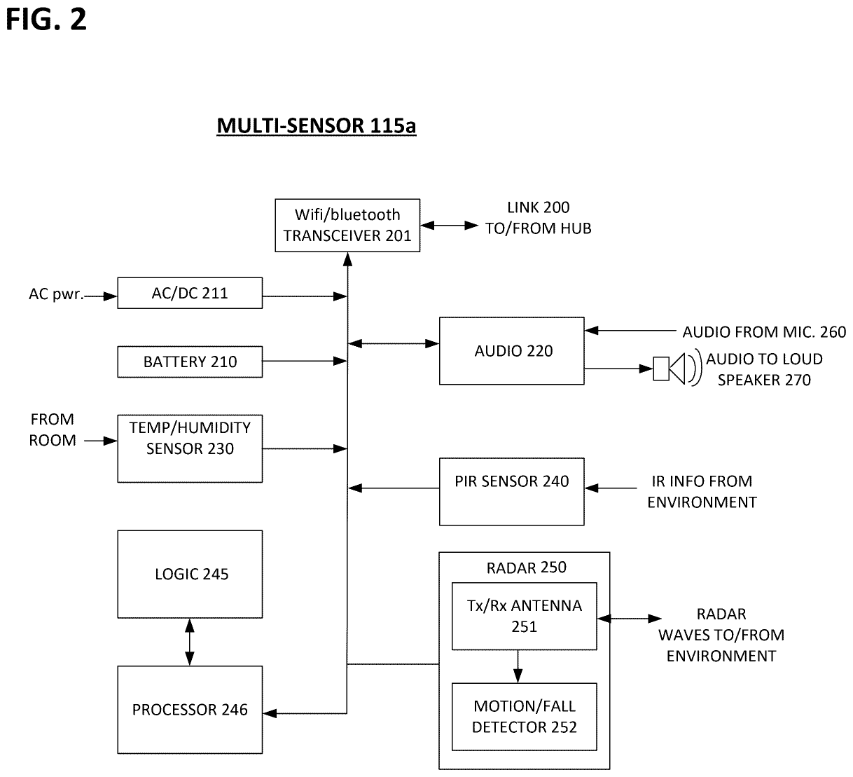

As described earlier, the system 100 can use information detected by functionality comprising each sensor, 115a to 115n, to determine a current location and status of an individual being monitored. FIG. 2 is a block diagram illustrating functionality comprising one of the sensors 115a. For the purposes of this description, the functionality of only one sensor labeled 115a is described here, however it should be understood that each of the sensors have substantially similar functionality. As previously described, each sensor is connected to the hub 110 over a wireless communication link, such as the link 200 in FIG. 2. The sensor has a Bluetooth or Wifi transceiver 201 operating to transmit and receive information to and from the hub over the link 200. As previously described, the sensor can be either battery (210) operated, or it can be plugged into a wall socket to receive AC power which is then transformed to DC power by an AC/DC converter 211. The sensor also has audio functionality 220 that operates to receive sound information from a microphone 260, and to send this information over the link 200 to the hub. This sound information can be an utterance (i.e., speech) generated by a monitored individual such as a command to activate a sensor proximate to the individual. Once the sensor is active, the audio function 220 can operate to monitor sound generated in the environment proximate to it. The sensor can also operate to receive voice information from a caregiver over the link 200 from the hub, and to play the voice information over a loud speaker 270. In this regard, the sensor operates as a full-duplex wireless communication device that allows the caregiver and the monitored individual to communicate. The sensor also has a passive IR (PIR) type sensor 240 and a milli-meter wave (MMW) radar device 250 that operate cooperatively to detect motion and the presence of the monitored individual in the room. The PIR sensor can operate on battery or line power which can transformed to be 5-12 VDC for example. The radar 250 can have a transmit/receive antenna 251 and it can have motion and/or fall detection functionality 252. In the event that the radar 250 has fall detection functionality, when it detects that the monitored individual has fallen, it will send a signal to the hub indicative of the fall. Otherwise, the motion detector comprising the radar sends motion information to the hub. Finally, each sensor has processing functionality 246 that operates, in conjunction with logical instructions 245, to control certain aspects of the sensor functionality.

During a time that the sensor 115a does not detect movement within its field of view, the processor 246 can control the sensor to be in a low-powered or inactive state, during which time at least the radar 250 and the microphone, and possibly other functionality, are not powered and are inactive. At a time that the PIR sensor 240 detects movement, it generates a “movement detected” signal that is received by the processor 246 which results in the processor 246 controlling the MM radar and the microphone to transition to an active state. The processor controls the sensor to remain in the active state during a time that the PIR or the MM radar detects movement. During the time that the sensor is active, it can operate to periodically generate movement information and sound information that the processor 246 detects and which is sent to the hub. During the time that the processor 246 detects that the MM radar is active and detecting movement, it controls the sensor to remain in the active state. On the other hand, after some selected period of time the processor determines that the radar no longer detects movement, it can control the sensor to transition to the lower powered state and the radar becomes inactive.

During a time that the radar 250 is in an unpowered state, the PIR sensor 240 can continually monitor the environment proximate to it for movement of an IR source. The PIR sensor 240 uses only a small amount of power until it detects movement of an IR source. When the PIR detects movement, it can generate a “movement detected” signal that is detected by the processor 246 and the hub, and the processor can respond by controlling the radar 250 to transition to a powered state. As described previously, the radar can be controlled to transition to the low-powered state when it is determined by the processor that the radar is no longer detecting movement.

With continued reference to FIG. 2, in the event that two or more sensors are located in the same space, and if the field-of-view of their radar 250 overlaps, it is necessary for the system 100 to determine which sensor is in the best position to detect activity associated with the monitored individual. In this case, the hub can maintain a count of the number of times a PIR 240 associated with each sensor generates a “movement detected” signal, and it can use this count information to determine which of two or more sensors in a room (if there are multiple sensors in a room), or which room sensor (if some or all the rooms have only one sensor), should be active at any one time. So, for example, if a particular room has more than one sensor, and the hub receives more motion detection signals from a first sensor than any of the other sensors in the room, then the radar on this first sensor can be controlled to be active. Alternatively, if each of multiple rooms has at least one sensor, and the hub receives more signals from a sensor located in a first room than a sensor in another other rooms, then the radar comprising the sensor in the first room can be controlled to be active. The radar 250 comprising a particular sensor can be controlled (by the hub or the sensor processor 246) to operate continually while it detects movement (i.e., the individual being monitored), or the radar can be controlled to be active for some predetermined/programmed period of time. When movement is no longer detected, or the programmed powered on-time lapses, the radar comprising a particular sensor can be controlled to be inactive, or transition to an unpowered state.

According to another embodiment, each one of a plurality of sensors in the same or different rooms can comprise an ad-hoc network in which each sensor can periodically transmit a signal to one or more of the other sensors that comprises the number of times their PIR detects movement, and then the sensor having the highest number of detected motion (which is indicative that that sensor is in the best location to monitor an individual's activity) can arbitrate with the other sensors to be an active sensor.

With further reference to the radar 250 in FIG. 2, the motion information detected by the radar can be transmitted to the hub 110 (described later with reference to FIG. 3) which in turn can transmit the motion information to the server 160 that has functionality which operates to analyze this information and determine what type of motion a monitored individual is exhibiting. Alternatively, the radar 250 can have the functionality needed to detect a falling motion, in which case the radar can send a message to the hub that indicates a fall has been detected, and the hub can take appropriate action which can include sending a message to the caregiver alerting them to the possibility that the monitored individual has fallen.

As described earlier with reference to FIG. 2, each sensor 115A-115n can have a microphone 260 to detect sound. The sensor can transmit sound captured by the microphone to the hub 110 which can use the sound to determine that the monitored individual in currently in a particular location or room within their house, or to determine a status of the monitored individual. The hub can then use this sound information to activate a sensor that is in close proximity (i.e., closest sensor) to the monitored individual. By the same token, the hub can use the sound information to power-down sensors that it determines are not in the same room as the individual, or that are not the closest sensor(s) to the monitored individual. This control by the hub of the sensor activity can ensure that the system provides high-quality, full-duplex audio communication between the caregiver and the monitored individual without unnecessarily draining the batteries. Alternatively, the sensor processor 246 can detect that sound information is captured by the microphone and control the sensor to transition to the active state, or conversely control the sensor to transition to the low-powered state is it determines that sound information is not detected for some selected period of time. Accordingly, the powered state of a sensor can be controlled to be in a low-powered or powered state if either or both of sound or movement is detected within the FOV of the sensor.

FIG. 3 is a block diagram illustrating functionality comprising the central hub 110. As described previously, the central hub or hub 110 generally operates, under control of a processor 346, to receive information from the multi-sensors 115a to 115n, to process this information, and to generate and send and to receive messages to and from a caregiver in the event that it can determine from the information that a monitored individual may need help and the caregiver elects to establish a session with the hub. Also, the hub can operate to control certain operational states of the multi-sensors, such as controlling one or more sensor functions to change a powered state. The hub can have a network interface card (NIC) 300 that supports communication between it and the multi-sensors (i.e., Bluetooth link 320), and between it and the caretaker (i.e., Internet link 310) or the server 160. The hub has a processor 346, memory 340, and audio functionality 355. The memory 340 has sound processing functionality 344 that can operate to process sound information received from the sensors to determine what type of sound (i.e., speech or non-speech type sound) the sensor receives and to recognize certain words or combination of words uttered by someone proximate to the sensor. For example, sound can be classified as being generated by a human (i.e., the monitored individual or some other individual), or sound can be classified as being generated by something else (i.e., generated by an appliance, television, radio or other entertainment type sound, animal sound, or a dangerous sound such as a gun shot, or glass breaking). Some sounds generated by a human can be classified as dangerous sound and lead to trouble for the monitored individual (i.e., sound of the monitored individual falling or a gunshot or threatening speech), and cause the hub to generate an alert message that is sent to a caregiver, and some sounds can be classified as being benign and simply ignored. Depending upon the class of sound detected, or the words recognized by the hub, the hub can respectively generate and send a message to the caregiver or to the server 160. A message to the caregiver can be indicative that the monitored individual may need some sort of attention, which can lead to the caregiver calling the individual or the caregiver escalating the process by calling for an emergency response (i.e., nurse, ambulance, police, etc.). Alternatively, when the hub recognizes a word or word combination, this can trigger logic comprising the hub to send any subsequent sound detected by the sensor to the server 160 for further processing. For example, if the hub recognizes a “wake up” word, then sound subsequently detected by the sensor can be transmitted to the server for processing, and depending upon the results of this processing, the server can generate and send a message to the caregiver.

With further reference to FIG. 3, the sound processing functionality 344 can be comprised of a neural network (NN) that is trained to detect different words or word combinations, and different classes or types of sounds, and the output of the NN can be either sent to caregiver message generation logic 342 or to the server 160. Message generation logic 342 is comprised of logical computer instructions maintain in non-volatile memory that when operated on by the processor 346 can cause a message to be generated and sent to a caregiver. The message to the caregiver can be a general message, or it can be a message that is indicative that the monitored individual needs some particular type of assistance.

As described earlier, the hub 110 in FIG. 3 can operate to receive motion/movement information from the MM wave radar or the fall detector. In the event the fall detector determines that a monitored individual has fallen, a fall signal is sent to the caregiver. Alternatively, motion information received by the hub from the MM wave radar can be transmitted directly to the server 160 for processing. The processor 346 in conjunction with the logic 341 controls all of this activity. The server 160 is comprised of functionality that can perform statistical analysis on the motion information it receives from the hub. The analysis is directed to identifying motion that corresponds to particular types of activity, such as walking, sitting, sleeping, eating to name only a few. After the statistical analysis is performed, the server can operate to detect the different types of movement, and depending upon the movement type can generate and send a message to the caregiver alerting them to the type of movement exhibited by the monitored individual.

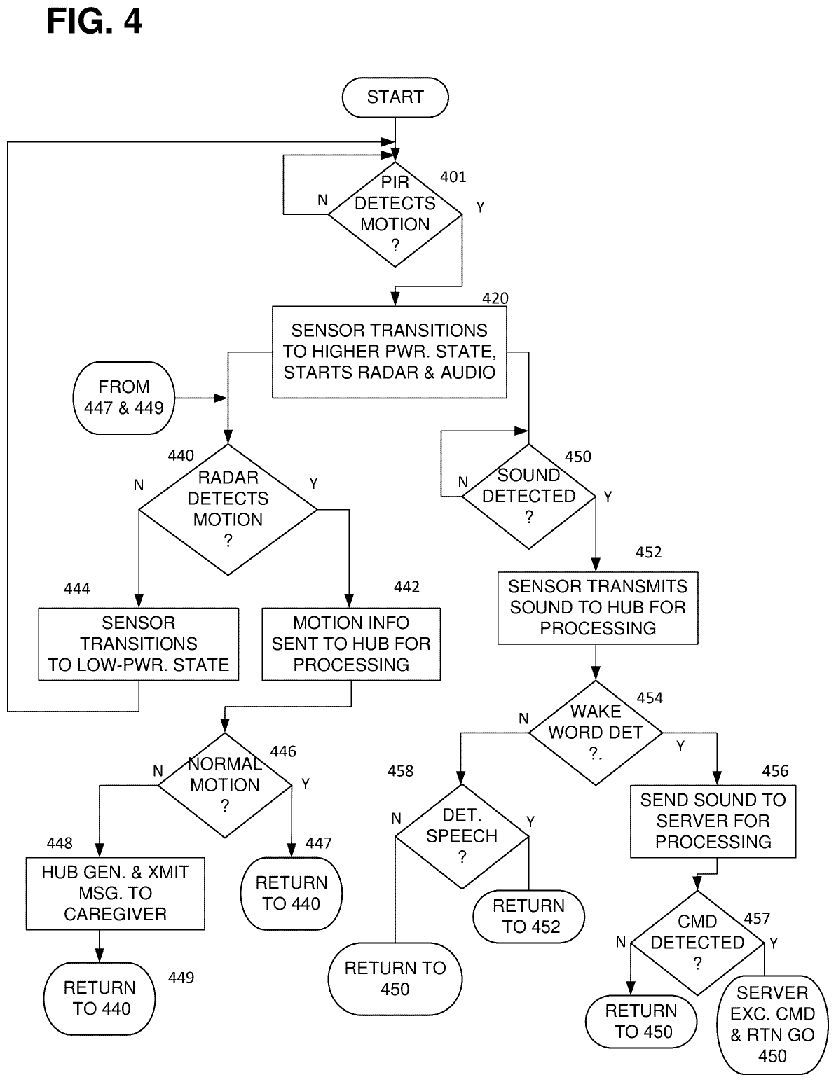

FIG. 4 is a logical flow diagram illustrating the operation of the multi-sensor 115a according to an embodiment in which an area or room has a single sensor. At 400 the sensor 115a is inactive and in a low-powered state in which the radar 250 and the audio functionality 220 are not operational. At 401, if the PIR on the sensor 115a detects IR movement, it generates a “movement detected” signal that the sensor processor detects and controls the sensor 115a, at 420, to transition to a higher-powered state (active state) and turns on the radar and the audio functions. On the other hand, if the PIR does not detect movement it continues to look for movement at 401. Once the sensor has transitioned to the active state, the audio 220 and the radar 250 operate to respectively detect sound and to detect movement. The sound can be generated by an individual being monitored or it can be generated by some other source proximate to the sensor 115a. The movement can also be generated by an individual being monitored or by another individual proximate to the sensor. In the event that the sensor detects movement by multiple individuals, the radar can be controlled by the processor to be inactive. If at 440 the radar detects motion, the processor can control the transceiver at 442 to send the motion information to the hub/server for processing. On the other hand, if at 440 the radar does not detect motion, the sensor is controlled to transition to a low powered state and the process returns to 401. At 446, the radar information processing 340 running on the hub or the server 160 examines the motion information to determine if the motion is representative of normal or abnormal motion. Alternatively, if the radar 250 is implemented to have fall detection capability, then it generates a signal that the processor sends to the hub which is indicative that the monitored individual has fallen (i.e., detects abnormal motion). If at 446 the hub or the server determines that the motion is normal, then the process returns to 440, otherwise at 448 the caregiver message logic 345 comprising the hub or the server can determine that a message should be generated and transmitted to the caregiver indicating that the monitored individual may have fallen and is in need of assistance.

Continuing to refer to FIG. 4, and as described earlier, when the sensor transitions to a higher-powered state at 420, both the radar 250 functionality and the audio 220 functionality can be activated. If at 450 the microphone 260 captures sound information, this sound can be transmitted by the sensor to the hub classification function 330 for processing at 452. As described previously with reference to FIG. 3, the sound classification function 330 can identify different types of sound as speech or non-speech type sound and it can identify certain words or combinations of words. If at 454 it is determined that a wake word is detected, the process can proceed to 456, and all subsequent sound (or some programmed amount of sound) can be sent to the server 160 for processing, otherwise at 458 the sound can be sent to the hub for classification. If at 456 the server determines that the sound includes a command, the server can execute the command and the process returns to 450. Commands uttered by a monitored individual can include, but at not limited to, such combinations of words as “I have fallen and need help”, or “I can't move and need help”, or simply “I need help”. If at 454 a wake word is not detected, then the process proceeds to 458, and the hub determines whether speech is detected. If so, then the process returns to 452, otherwise the process returns to 450.

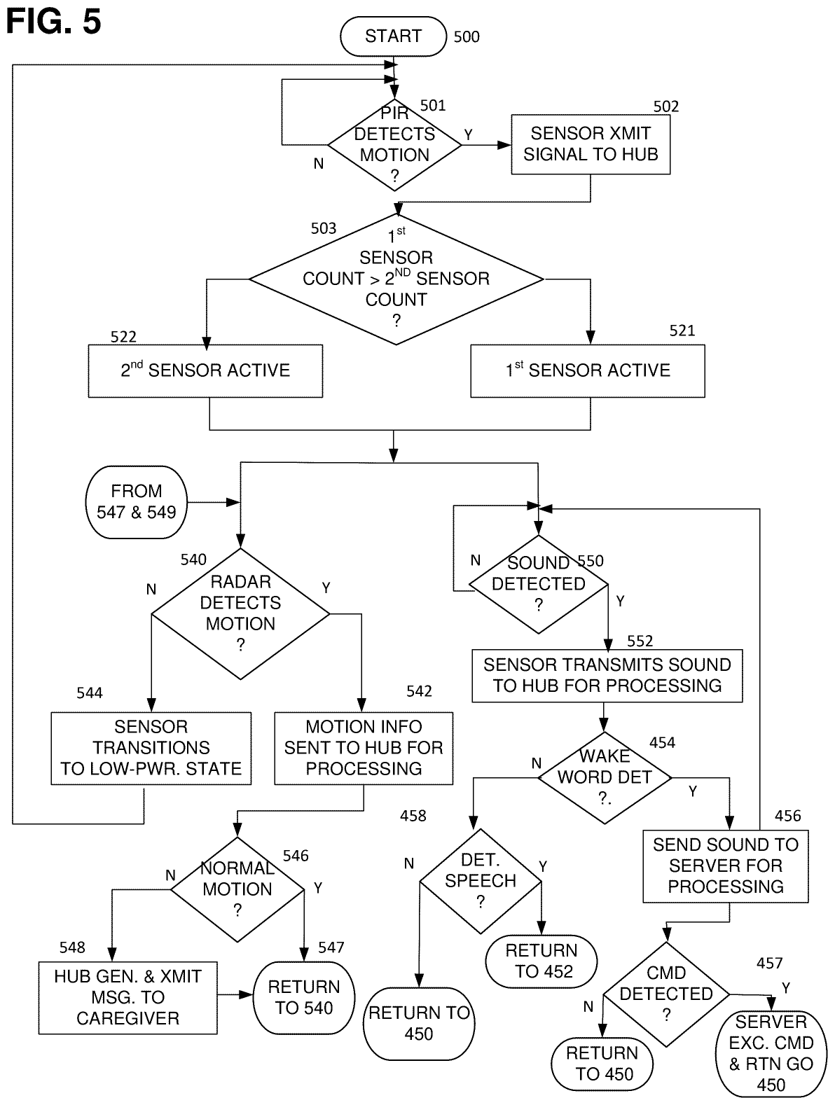

As described previously with reference to FIGS. 1B and 1C, depending upon the dimensions and shape of a space/room being monitored, it may be necessary to place two or more sensors in the room to provide adequate radar coverage and to ensure that the microphone and loud speaker 220 on at least one of the sensors is proximate to a monitored individual. FIG. 5 is a logical flow diagram showing the operation of the system 100 having two or more sensors in one room. According to the embodiment described in FIG. 5, a PIR on one of the sensors in the room may detect movement at the same time, or at different times within some selected period of time, as a PIR on another one of the sensors. In this case, the system can determine which one of the sensors is closer to a monitored individual by maintaining a count of the number of times a PIR generates a “movement detected” signal. The movement detected signals from each sensor can be transmitted to the hub where a separate count for each sensor is maintained, or the count can be maintained locally by each sensor. Regardless of the manner in which the count is maintained, the sensor with the highest count can be selected for activation to monitor the individual. If the hub performs the selection process, it can select the sensor having the highest number of “movement detected” signals and activate this sensor, or if the sensors can establish an ad-hoc network among themselves, then they can conduct an arbitration process, and the sensor that wins the arbitration can automatically transition to a higher-powered state.

The process described in FIG. 5 is substantially the same as the one described with reference to FIG. 4, with the exception that the hub 110 controls which sensor is active at any particular point in time. For the purpose of the FIG. 5 description, a room or space has two sensors, a first and a second sensor. At the start 500 both the first and the second sensors are in an inactive state until at 501 a PIR on either or both of the sensors detects motion, at which time a “motion detection” signal from that sensor, or sensors, is transmitted at 502 to the hub 110. The hub maintains a count of the number of motion detected signals are received from each sensor, and if at 503 the hub determines that the first sensor has detected more motion than the second sensor, then the hub controls the first sensor to become active, otherwise the hub controls the second sensor to become active. The logical process subsequent to the hub activating one of the sensors is substantially the same as that after 420 in FIG. 4, and so will not be described here.

The forgoing description, for purposes of explanation, used specific nomenclature to provide a thorough understanding of the invention. However, it will be apparent to one skilled in the art that specific details are not required in order to practice the invention. Thus, the forgoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. (canceled)

2. (canceled)

3. (canceled)

4. (canceled)

5. (canceled)

6. (canceled)

7. (canceled)

8. (canceled)

9. A method for controlling a wireless communication device, comprising:
each one of a plurality of wireless communication devices remain in an inactive state while a passive motion detection device, comprising each one of the wireless communication devices, does not detect motion;
a first one of the plurality of the wireless communication devices transitioning to an active state, and enabling an active motion detection device, comprising the first wireless communication device, when motion is detected by the passive motion detection device;
enabling an audio communication function, comprising the first wireless communication device, when motion is detected by the active motion detection;
the audio communication function comprising the first wireless communication device remaining in the active state while an active motion detection device detects motion; and
the first wireless communication device transitioning to the inactive state and disabling the audio communication function and the active motion detection function when the active motion detection device no longer detects motion.

10. The method of claim 9, further comprising a passive motion detection device, comprising a second one of the plurality of the wireless communication devices, detecting motion and the second wireless communication device transitioning from an inactive state to an active state subsequent to the first wireless communication device transitioning to the inactive state.

11. The method of claim 10, wherein an audio communication function and a microphone comprising the second wireless communication device are enabled.

12. The method of claim 9, wherein a microphone comprising the first wireless communication device is not enabled while the first wireless communication device is in the inactive state.

13. The method of claim 12, wherein the first wireless communication device controls the microphone to be enabled to capture sound information when the first wireless communication device transitions to the active state.

14. The method of claim 13, wherein the first wireless communication device establishes a wireless link between it and a central hub and transmits the captured sound information to the central hub.

15. The method of claim 14, wherein the hub classifies the sound it receives from the first wireless communication device as speech, and determines that the speech comprises a wake word.

16. The method of claim 15, wherein the central hub transmits all sound information captured by the microphone to a server connected to the hub subsequent to recognizing the wake word.

17. The method of claim 16, further comprising the server processing the sound information to determine whether or not it comprises a recognizable command.

18. The method of claim 17, further comprising the central hub generating and sending a message to a caregiver if the server recognizes a command.
